(19) United States
(12) Patent Application Publication
(10) Pub. No.: US 2026/0090890 A1
(43) Pub. Date: Apr. 2, 2026

(54) DEVICE FOR ANCHORING A SHOULDER PROSTHESIS IN A REVERSE CONFIGURATION, BONE FIXING SCREW AND ANCHORING SYSTEM

(71) Applicant: MEDACTA INTERNATIONAL SA, CASTEL SAN PIETRO (CH)

(72) Inventors: Andrea ROSA, CASTEL SAN PIETRO (CH); Francesco SICCARDI, CASTEL SAN PIETRO (CH); Massimiliano BERNARDONI, CASTEL SAN PIETRO (CH)

(21) Appl. No.: 19/114,921

(22) PCT Filed: Sep. 28, 2023
(86) PCT No.: PCT/IB2023/059675

§ 371 Date: Mar. 25, 2025

(30) Foreign Application Priority Data
Sep. 30, 2022 (IT) 102022000020094

(51) Int. Cl. A61F 2/30; A61B 17/86; A61F 2/40

(52) U.S. Cl. CPC A61F 2/30749; A61B 17/8625; A61F 2002/30433; A61F 2002/30774; A61F 2002/30784; A61F 2002/30901; A61F 2002/4085

(57) ABSTRACT

A device for anchoring a shoulder prosthesis in a reverse configuration includes a baseplate delimited at the bottom by a first at least partially convex surface, and at the top by a second concave surface, opposite the first surface, and peripherally by a third truncated-cone or cylindrical surface; and a central pin, protruding from the first surface at a first end thereof, and defining an axially pass-through inner duct having at least one main diameter. The central pin has a second free end, opposite the first end. The duct has a helical cavity made in the inner wall delimiting the duct, open towards the inside of the duct and extending along the length of the duct. The inner duct has a secondary diameter smaller than the main diameter, and the helical cavity has a diameter larger than the main diameter of the inner duct.

### TECHNICAL FIELD

The present invention relates to a device for anchoring a shoulder prosthesis in a reverse configuration, a bone fixing screw to be used with the mentioned anchoring device and a system for anchoring a shoulder prosthesis in a reverse configuration.

A conventional (or anatomical) shoulder prosthesis attempts to reproduce, and thus mimic, the normal anatomy of the shoulder: a polyethylene “cup” is mounted in the bone pocket of the scapula (glenoid), and a “ball” made of a biocompatible metal material is connected to the upper part of the arm bone (humerus).

In a reverse prosthesis, the cup and ball are reversed. The ball (glenosphere) is fixed to the glenoid cavity and the cup is fixed to the upper end of the humerus.

Reverse shoulder configuration, therefore, refers to a total shoulder arthroplasty system that reverses the normal biomechanics between the scapular and humeral components: the joint action mechanism is reversed and the glenoid cavity is replaced with a glenosphere, while the sphericity of the humeral head is replaced with a polyethylene concavity that articulates with the glenosphere.

The device that is the subject of the present invention is used as a system for anchoring the implant to the glenoid cavity, in particular as a system for anchoring a baseplate to which the glenosphere is associated.

### BACKGROUND ART

There currently exist several devices for anchoring a shoulder prosthesis in a reverse configuration.

Some devices of the known type include a baseplate provided with a central pin, which can be inserted inside the glenoid cavity, in a single-piece configuration. The pin may be cylindrical in shape and may be impacted inside a hole previously made in the glenoid cavity. In this case, the baseplate is impacted on the glenoid cavity and an additional central screw cannot be used to provide the implant with more compression and stability.

Alternatively, the glenosphere support is provided with a screw, integral with the baseplate, which is screwed into the glenoid cavity.

This solution has the disadvantage that it does not allow the baseplate to be positioned according to a planned orientation, since the baseplate is screwed down until it contacts the glenoid cavity. The baseplate has four peripheral holes, arranged at 90° to each other, for inserting connection screws. Since the holes are made after the baseplate has been positioned, it may happen that the holes coincide with a position where there is little bone or the quality of the bone is poor, with the consequence that the baseplate cannot be firmly fixed to the glenoid cavity.

In addition, these types of prostheses may have a baseplate provided with asymmetrical shims that are used to compensate for any lack of bone thickness. Therefore, if I screw the baseplate until it comes into contact with the glenoid cavity, it may happen that the shim, or wedge, is not positioned where required, thus creating an incorrect coupling of the prosthesis with the abutment surface.

Another solution used in the prior art provides a baseplate provided with a central pin having a substantially cylindrical shape. The pin is pierced in the middle. After impacting the baseplate into the bone, hence after inserting the pin into the hole previously made in the glenoid cavity, a screw is passed through the central cavity of the pin. The screw can rotate independently of the baseplate, screwing itself into the bone. This system with a central pin and modular screw inserted after the baseplate has been impacted has the drawback of having a pin with a central pass-through hole, which has a diameter larger than that of the thread of the screw to allow insertion thereof. Therefore, it is a bulky device that necessarily requires a good base bone implant, with a wide thickness to allow a pin, having a certain diameter dimension, to be housed.

Finally, there is a further type of prosthesis, similar to the one described above, which comprises a support base (baseplate) having a central pin that is substantially cylindrical in shape and internally pierced.

A modular central screw can be inserted into the hole of the pin before the baseplate is positioned and impacted into the bone, unlike the embodiment described above, where the screw is inserted with the baseplate already positioned in the bone.

In this embodiment, in fact, the screw is not inserted through the baseplate along the pin, but from the tip of the central pin (the one that is first inserted into the bone) back towards the baseplate.

The disadvantage of such a solution is that the surgeon cannot decide whether to use the central screw after the baseplate has impacted on the basis of the stability of the implant, but is forced to fix it by means of the central screw as well. Furthermore, in the case of prosthesis revision, it is not possible to unscrew the screw before removing the baseplate, resulting in complications during revision surgery.

An object of the present invention is to overcome the drawbacks of the prior art.

In particular, the object of the present invention is to propose a device for anchoring a shoulder prosthesis in a reverse configuration, which has a pin having limited dimensions, such that it may be implanted even in situations where there is a shortage of bone, and such to have the possibility of positioning the pin along the spine of the scapula, maximising the stability of the implant.

A further object of the present invention is to create a device for anchoring a shoulder prosthesis in a reverse configuration, which has a screw independent of the support base, so that the support base itself can be freely oriented to best position the peripheral screw seats and any correction wedges or shims.

An object of the present invention is also to provide a device for anchoring a shoulder prosthesis in a reverse configuration, which allows the screw to be inserted through the baseplate after impaction, so as to decide, after impaction, as to whether the screw is required, on the basis of the stability of the implant.

Furthermore, the object of the present invention is to provide a device for anchoring a shoulder prosthesis in a reverse configuration that allows, in the event of revision, the screw to be unscrewed before removing the baseplate, thus simplifying the implant removal.

A further object of the present invention is to propose a bone fixing screw to be used with the aforementioned anchoring device to allow it to be properly fixed to the shoulder.

Furthermore, and not least, the object of the present invention is to provide a system for anchoring a shoulder prosthesis in a reverse configuration that overcomes the drawbacks of the prior art.

These and other objects and advantages are achieved by a device for anchoring a shoulder prosthesis in a reverse configuration as shown in the attached claims.

### SUMMARY

A first aspect of the present invention provides a device for anchoring a shoulder prosthesis in a reverse configuration, comprising a baseplate delimited at the bottom by a first, at least partially convex surface, adapted to interface with a glenoid cavity, at the top by a second concave surface, opposite to the first convex surface, and peripherally by a third truncated-cone or cylindrical surface. A central pin is protruding from the first, at least partially convex surface and has a pass-through inner axial duct. The central pin has a first end connected to the baseplate and a second free end, opposite the first end. The inner axial duct has a helical cavity obtained in the inner wall delimiting the duct itself, which is open towards the inside of the duct and extending along the full length of the duct, so as to place the first end in fluid communication with the second end of the central pin.

Advantageously, the inner duct has a secondary diameter smaller than the main diameter. Preferably, the helical cavity has a diameter greater than the main diameter of the inner duct

The helical cavity has a pitch and profile designed to be coupled with a pitch and profile of a screw thread that can be screwed into the duct.

The depth of the helical cavity is also equal to the thread height of the fixing screw.

The baseplate has a central through-hole, made at the pin, communicating with the duct inside the pin.

The first end of the pin is connected to the baseplate and opens externally into the first concave surface at that hole, while the second free end of the central pin, axially opposite to the first, is open externally.

The inner duct has at least one threaded portion having a pitch and height different from the helical cavity, so that it can be coupled with a second threaded screw that can be inserted through the first end of the central pin, adapted to fix a glenoid cavity.

The change in the diameter of the duct takes place in an intermediate zone between the second surface of the baseplate and the second end of the central pin, thus in an intermediate zone between the first and second end of the central pin.

The duct has a greater main diameter extending into the portion between the baseplate and the intermediate zone, and a smaller secondary diameter extending into the portion between the intermediate zone and the second end of the central pin. The pin has a cylindrical shape and a corrugated outer surface.

The baseplate has at least two pass-through holes which place the first, at least partially convex surface, in communication with the second concave surface.

The first surface shows, at least partially, raised wedge-shaped portions.

In a second aspect, the present invention provides a bone fixing screw that can be inserted into a device for anchoring a shoulder prosthesis in a reverse configuration. The screw comprises a cylindrical body, a tip located at an end of the cylindrical body, a head located at a second end of the cylindrical body, opposite the first end, and a thread extending helically along the entire axial extension of the cylindrical body. The screw head has an outer diameter greater than the outer diameter of the cylindrical body and smaller than the outer diameter of the thread.

The head has a cylindrical shape with no protruding portions; the head is connected to the cylindrical body by a tapered portion.

The tip of the screw has at least one notch with a sharp edge in order to tap as it is inserted, thus avoiding preliminary tapping.

In a third aspect, the present invention provides a system for anchoring a shoulder prosthesis in a reverse configuration comprising an anchoring device, as described and claimed in the present invention, and a fixing screw, as described and claimed in the present invention.

The anchoring system requires that the helical cavity, which is obtained inside the duct of the pin, has a pitch, profile and depth designed to be coupled with a pitch, profile and height of the thread of the fixing screw that can be screwed into the duct.

### BRIEF DESCRIPTION OF THE DRAWINGS

The present invention will be made clearer by the following detailed description, with reference to the accompanying drawings provided by way of example only, wherein:

FIG. 1 shows a perspective view, from above, of a device for anchoring a shoulder prosthesis in an reverse configuration according to the present invention and in a first configuration;

FIG. 2 shows a side view of the device that is the subject of the present invention, according to a second embodiment;

FIG. 3 shows a lower perspective view of the device that is the subject of the present invention, according to a third embodiment;

FIG. 4 shows a side view of the device that is the subject of the present invention, according to a fourth embodiment;

FIGS. 5a and 5b show, respectively, a perspective view and a side view of a bone fixing screw according to the present invention, which can be coupled to the device for anchoring a shoulder prosthesis in a reverse configuration according to the present invention;

FIG. 6 shows a section side view of the system for anchoring a shoulder prosthesis in a reverse configuration according to the present invention, in accordance with the first shown configuration;

FIG. 7 shows a section side view of the system for anchoring a shoulder prosthesis in a reverse configuration according to the present invention, in accordance with the third shown configuration;

FIG. 8 shows a perspective view of a shoulder prosthesis in a reverse configuration that is fully assembled on the anchoring system that is the subject of the present invention;

FIG. 9 shows a section side view of the prosthesis shown in FIG. 8.

### DETAILED DESCRIPTION

In the above-mentioned figures, a device for anchoring a shoulder prosthesis in a reverse configuration in accordance with the present invention has been globally referred to as 1.

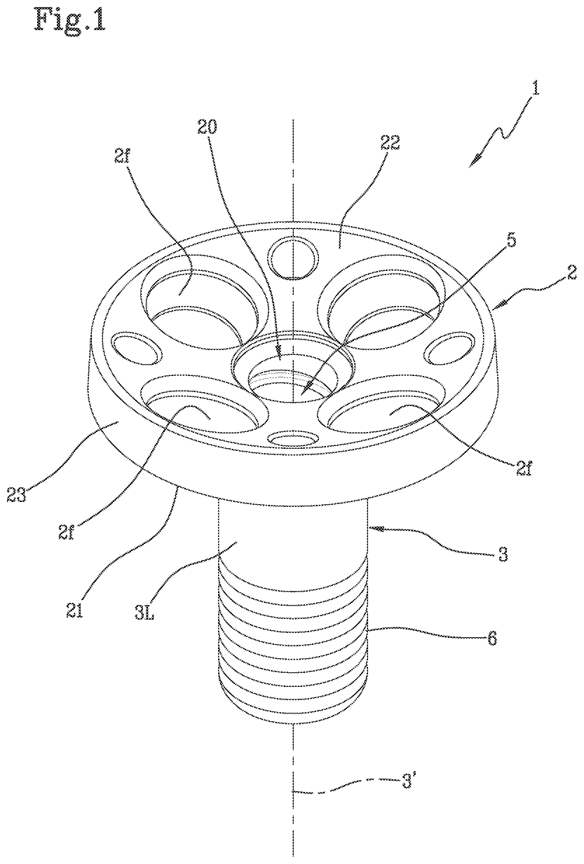

Referring in particular to FIGS. 1 to 4, which will be shown in detail hereinafter, the anchoring device 1 comprises a baseplate 2, and a pin 3 protruding centrally from such a baseplate 2.

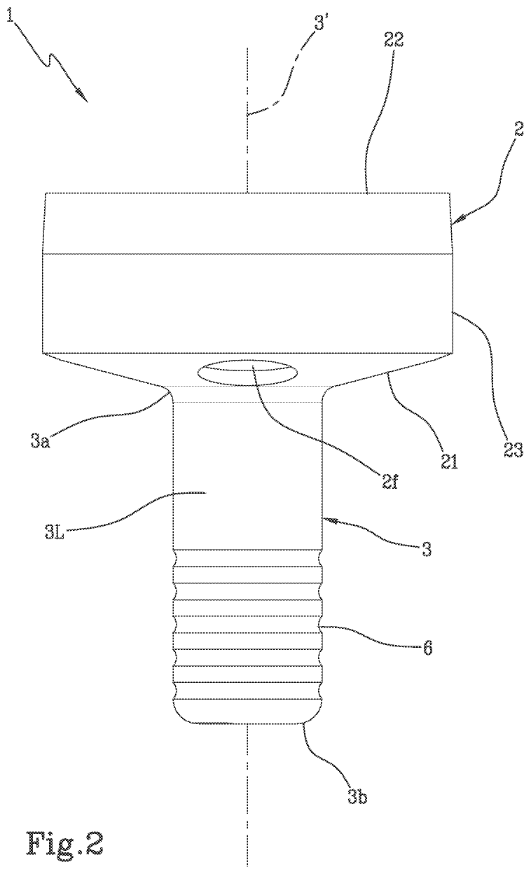

The baseplate is, in fact, a baseplate delimited at the bottom by a first surface 21 at least partially convex, adapted to interface with a glenoid cavity, and at the top, by a second concave surface 22 opposite the first convex surface 21. Peripherally, or laterally, the baseplate 2 is delimited by a truncated-cone or cylindrical third surface 23, which may have different thicknesses depending on the required configuration to be implanted. Two examples of the different thicknesses that the baseplate can have, and therefore of the height that the third surface 23 can have, are shown in FIGS. 1 and 2, which differ mainly in the height of the third side surface 23 delimiting the baseplate 2: lower in FIG. 1 than in FIG. 2. In these Figures, the configuration involving a third cylindrical side surface 23 is shown, but, as mentioned above, this surface can also be truncated cone-shaped.

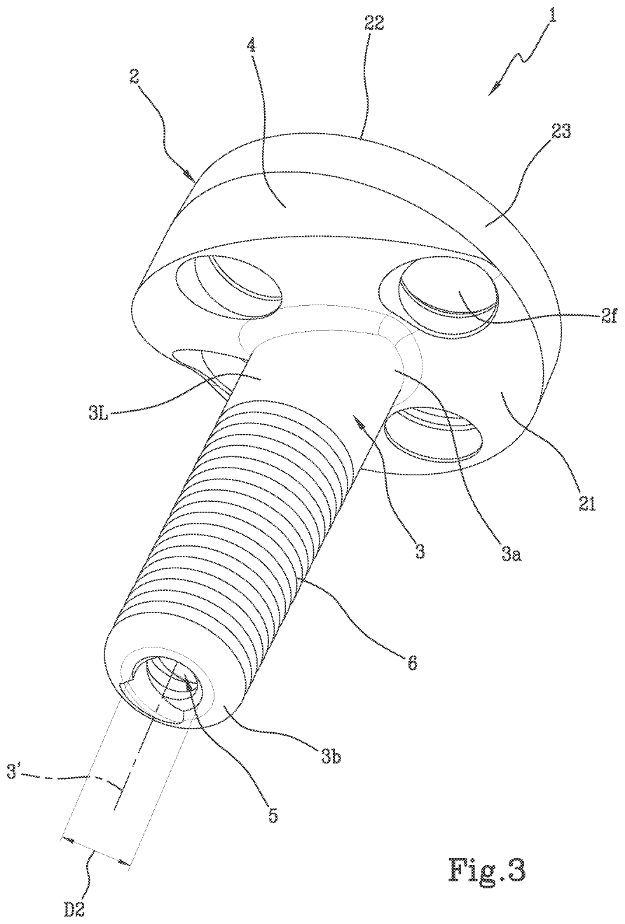

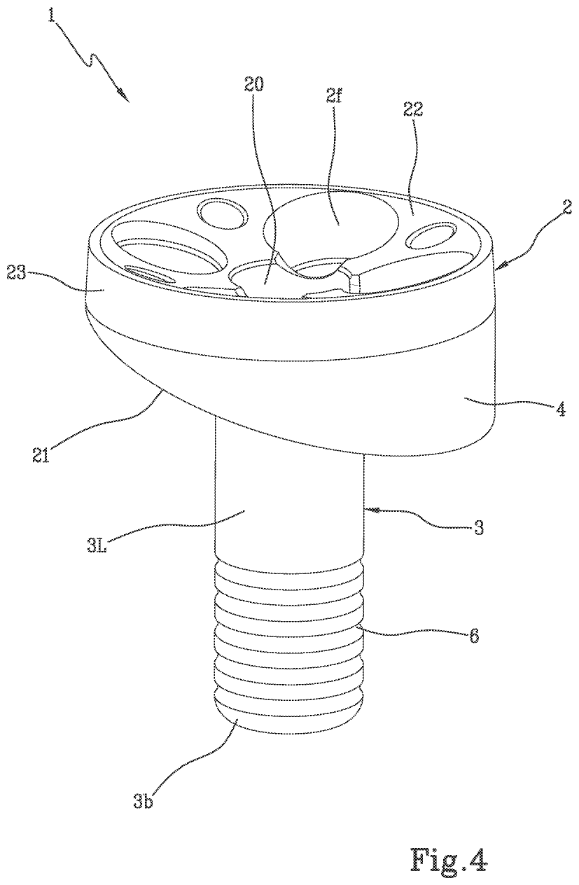

The first surface 21 is at least partially convex since, as shown in FIGS. 3 and 4, corresponding to as many possible alternative configurations, it may have raised wedge-shaped portions 4. This first surface 21, in fact, is the surface interfacing with the glenoid cavity and may be completely convex (as visible in FIGS. 1 and 2) or have partial (as in FIG. 3) or total protrusions (as in FIG. 4) specifically made according to the patient's anatomy, which correct the course of the lower surface of the baseplate 2, in order to compensate for any bone deficiencies. The lower surface of the baseplate (i.e. the first surface 21 of the baseplate) must match and settle completely on the glenoid cavity surface, without any voids or protrusions that could affect the correct positioning of the implant and thus its stability.

The central pin 3 is preferably cylindrical and protrudes from the first lower surface 21 of the baseplate 2, along a straight longitudinal axis 3′.

The central pin 3 has a first end 3a, or distal end, connected to the baseplate 2, and a second free end 3b, or proximal end, opposite the first end 3a.

The terms distal and proximal refer to the parts of the device respectively furthest and closest to the patient's body, with particular reference to the patient's heart.

The anchoring device is centrally hollow and therefore has, along its entire axial extension 5a, an inner duct 5 passing through and extending, for the entire longitudinal extension of the device, from the second surface 22 of the baseplate 2 to the free end 3b of the pin 3.

The duct 5 has a cylindrical cross-section and has an inner main diameter D1, which may be constant over the entire extension of the duct or may reduce to a secondary diameter D1′ in an intermediate zone along the extension of the duct 5. In the latter case, the inner duct 5 has a constant inner diameter equal to the main diameter D1 for almost the entire extension of the duct itself, which extends from the second surface 22 to an intermediate zone 30, placed between the first distal end 3a and the second proximal end 3b, and then undergoes a slight reduction to a secondary diameter D1′, slightly smaller than the main diameter, which extends with a constant size from the intermediate zone 30 of diameter change to almost near the second proximal end 3b of the pin 3. At the second proximal end 3b, then, the inner diameter of the duct 5 narrows further, defining a diameter D2 smaller than the secondary diameter D1′ of the duct 5.

The pin 3 may have different lengths depending on the patient's anatomy and on the shape of the bone into which the pin is to be inserted. The outer diameter of the pin 3 may also differ in size from one device to another, still depending on the size of the patient's bone; however, since the present invention solves the problem of minimising the size of the pin 3 so as to limit as much as possible the amount of bone to be removed to position the anchoring device, the preferred design will always be an anchor device which has a pin 3 having an outer diameter as small as possible, compatible with the size of the bone.

The baseplate 2 has a central hole 20 at the pin 3, thus communicating with the duct 5 inside the pin 3. The central hole 20 then passes through the thickness of the baseplate 2 and places the first 21 and second 22 surfaces of said baseplate 2 into communication.

The baseplate also has a plurality of peripheral holes 2f, arranged around the central hole 20, for the insertion of additional fixing screws 14 that could be used to better fix the implant to the bone. The holes are at least two, preferably four, and are pass-through holes to connect the first at least partially convex surface 21 with the second concave surface 22.

The first distal end 3a of the pin 3 is connected to the baseplate 2 and opens externally into the first concave surface 21 at the aforementioned hole 20, while the second proximal end 2b of the central pin 3, axially opposite to the first, is free and open externally.

Externally, on the side wall 3L, the central pin 3 has a corrugated outer surface with a series of circumferential discharges 6 to increase friction with the hole made in the bone and into which the pin 3 is to be inserted.

Internally, the duct 5 has, on the inner wall 5L, a helical cavity 7 that is open towards the inside of the duct 5 and extending along the full length of the duct 5. This helical cavity 7 places the first end 3a in fluid communication with the second end 3b of the central pin 3.

The helical cavity 7 has a pitch, profile and depth or height designed to be coupled with a pitch, profile and height of the thread of a fixing screw 8 that may be screwed within the duct 5.

In other words, the helical cavity 7 is the negative of the thread of the fixing screw 8 that can be inserted inside the duct 5, to fix the anchoring device 1 to the shoulder.

The minimum diameter D3 of the helical cavity 7 is equal to the inner diameter of the duct (main diameter D1 or secondary diameter D1′ depending on where the minimum diameter of the cavity 7 is assessed), while the maximum diameter D4 of the helical cavity 7 is greater than the inner diameter of the duct 5. The main diameter D1 is greater than the secondary diameter D1′ and smaller than the diameter D4 of the helical cavity 7.

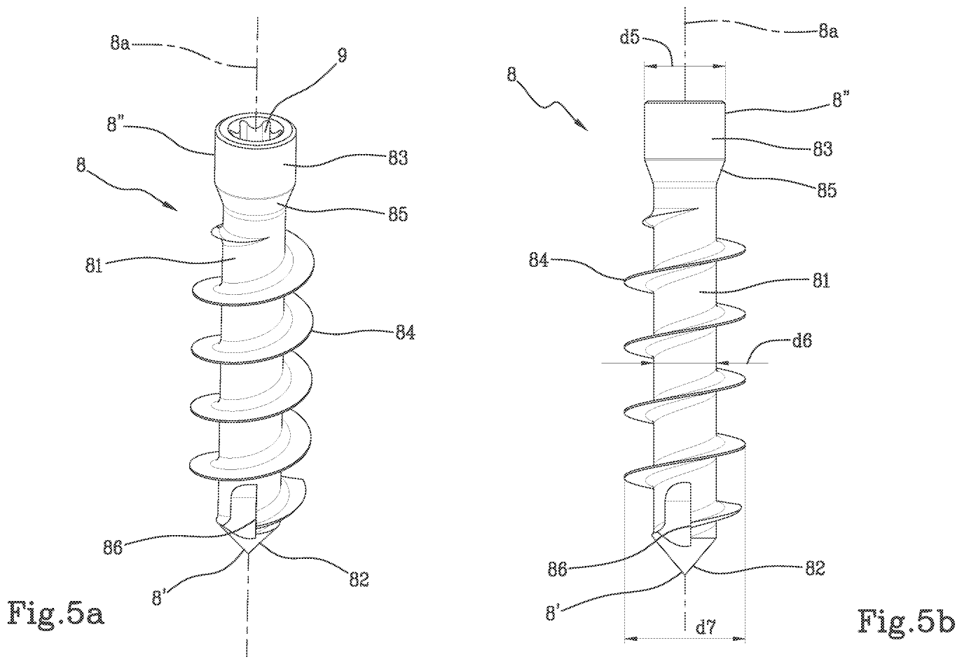

The fixing screw 8, shown in FIGS. 5a and 5b, comprises a cylindrical body 81 having a tip 82, placed at a proximal end 8′ of the cylindrical body 81, a head 83, placed at a second distal end 8″ of the cylindrical body, opposite to the first end 8′, and a thread 84, extending helically along the entire axial extension 8a of the cylindrical body 81.

The head 83 has an outer diameter d5 that is greater than the outer diameter d6 of the cylindrical body 81 and smaller than the outer diameter d7 of the thread 84.

The head 83 has a cylindrical shape with no protruding portions and is connected to the cylindrical body 81 by a tapered portion 85.

The tip 82 of the screw has at least one notch 86 having a sharp edge that allows the insertion of the screw into the bone without preliminary tapping in the bone: the notch, in fact, taps directly the bone as it is inserted.

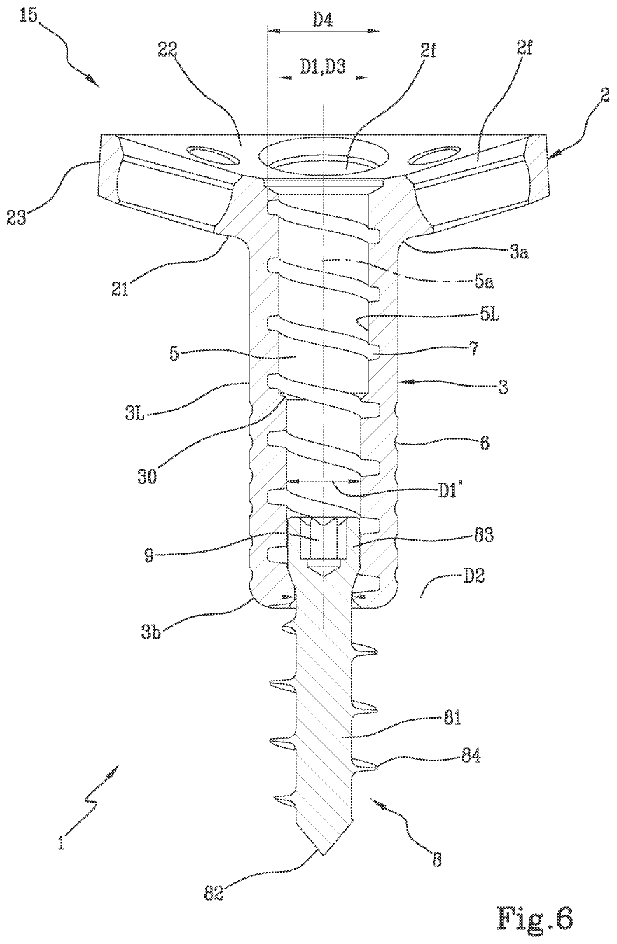

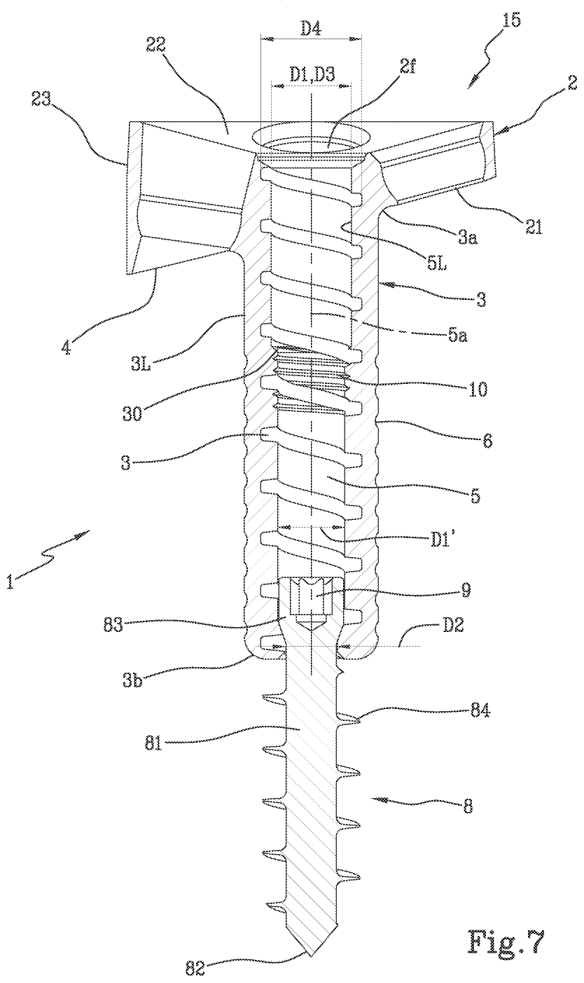

The diameter d5 of the head 83 of the screw 8 is equal to the inner secondary diameter D1′ of the duct 5, as visible in FIGS. 6 and 7. The outer diameter d6 of the cylindrical body 81 of the fixing screw 8 is equal to the smaller diameter D2 of the duct 5, placed at the second proximal end 3b of the pin 3. The outer diameter d7 of the thread 84 of the fixing screw 8 is equal to the maximum diameter D4 of the helical cavity 7 made in the wall 5L of the duct 5 inside the pin 3.

The head 83 of the fixing screw 8 has a housing 9 to be coupled with a fixing tool.

The length of the fixing screw 8 depends on the size and quality of the available portion of bone into which the screw is to be implanted.

The duct 5 inside the pin 3 also has, on the side wall 5L, at least one threaded portion 10 having a pitch and height different from those of the helical cavity 7, so that it can be coupled with a second threaded screw 11, which can be inserted inside the pin 3, through the central hole 20 of the baseplate 2. This second threaded screw 11 is adapted to fix a glenosphere 12 to the anchoring device 1.

The threaded portion 10 is arranged along the duct 5 and has a limited extension confined to a section of the duct 5.

In the attached figures, the threaded portion 10 is, by way of example only, shown in the section of duct 5 having a smaller secondary diameter D′. Alternative configurations may provide this threaded portion 10 inside the first section of the duct 5, the one having the greatest main diameter D.

The present invention also protects a system 15 for anchoring a shoulder prosthesis in a reverse configuration comprising the described anchoring device and an anchoring screw according to the present description.

The helical cavity 7 has a pitch and profile designed to be coupled with a pitch and a profile of the thread 84 of the fixing screw 8 that can be screwed into the duct 5 of the pin 3. In addition to the pitch and profile, the height or depth of the helical cavity 7 also corresponds to the height of the thread 84 of the fixing screw 8, so that the thread 83 of the fixing screw 8, during insertion of the screw 8 inside the duct 5, is entirely contained in the wall 5L delimiting the duct 5 of the pin 3: thereby, the pin 3 can have a limited transverse dimension compared to the pins of the prior art.

The anchoring system is used for shoulder arthroplasty operations, particularly in reverse configurations, to provide anchorage of the implant to the glenoid cavity.

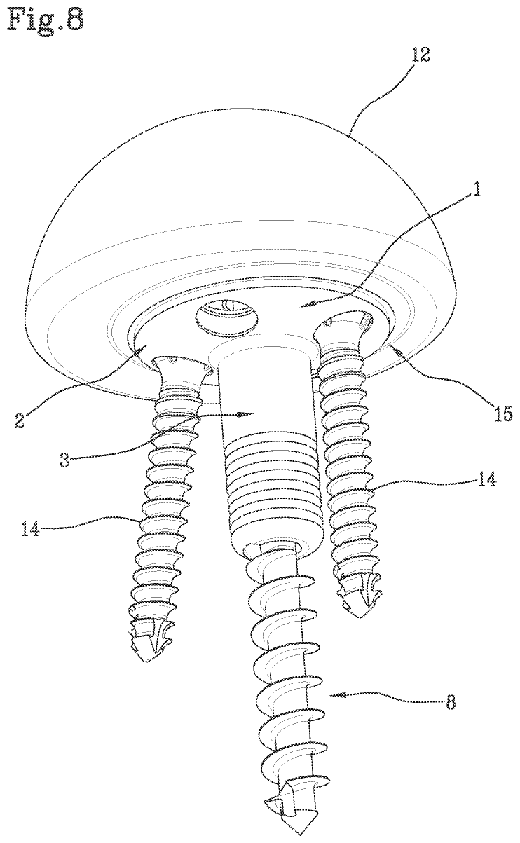

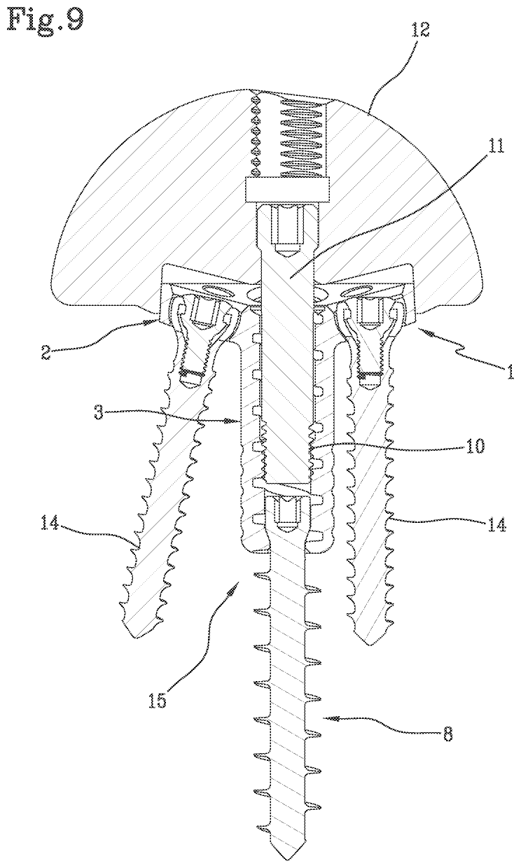

FIGS. 8 and 9 show a complete implant associated with the anchoring system that is the subject of the present invention.

The glenoid cavity is prepared to house the baseplate 2 of the anchoring device by the steps of milling the joint surface and preparing the central hole into which the pin 3 will be inserted. While impacting the baseplate 2 on the glenoid cavity, the surgeon can choose its orientation, rotating the plate about the longitudinal axis 3′ of the pin 3, to place the peripheral holes 2f where the bone is thicker and has a better quality, and to position the protrusions or wedge-shaped portions 4 of the first convex surface 21, if present, at bone deficiencies or reductions.

The corrugations of the outer wall of the pin and the circumferential discharges 6 allow for a greater interference with the walls of the hole made in the bone, as well as allows for better adhesion of the pin 3 to the bone tissue.

If the surgeon deems it necessary, on the basis of the stability of the implant or the preoperative planning, a central fixing screw 8 may be used to provide greater stability and compressive strength: a central hole is prepared using a special guide and tip, and the depth of the hole is assessed using a depth gauge to decide the length of screw to be used. The central fixing screw 8 is then inserted inside the scapula, passing through the central hole 20 of the baseplate 2.

The thread 84 of the fixing screw 8 fits into and runs through the entire helical cavity 7 made in the wall 5L delimiting the duct 5 inside the pin 3. A fixing tool allows to screw the fixing screw 8 all the way.

The surgeon continues with the preparation of the peripheral holes 2f and the insertion of the respective screws 13. After positioning a trial glenosphere, the operation can be completed by implanting the final glenosphere 12, inserting the second threaded screw 11 into the central hole 20 of the baseplate 2, to screw it to the threaded portion 10 present on the side wall 5L delimiting the duct 5 inside the pin 3.

The invention overcomes the drawbacks encountered in the prior art as it provides a device for anchoring a shoulder prosthesis in a reverse configuration that has a small-sized pin so that it can be implanted even in situations where there is little bone and the pin can be positioned along the spine of the scapula, maximising the stability of the implant.

Since the fixing screw is independent of the support base, it is possible to freely orient the support base itself in the way the surgeon deems best suited to the anatomy of the patient's bone, irrespective of the locations for the peripheral screws and any correction wedges or shims.

The insertion of the fixing screw through the baseplate after impaction gives the surgeon the opportunity to decide whether it is necessary to use the screw on the basis of implant stability, or whether the latter is not necessary because the implant, as it is, is already stable.

Inserting the fixing screw after the baseplate has been impacted allows, in the case of revision, the screw to be unscrewed before removing the baseplate, simplifying implant removal.

1. A device for anchoring a shoulder prosthesis in a reverse configuration, comprising:
a baseplate delimited at a bottom of the baseplate by a first at least partially convex surface, adapted to interface with a glenoid, at a top of the baseplate by a second concave surface, opposite the first surface, and peripherally by a third truncated-cone or cylindrical surface,
a central pin, protruding from said first surface, having an axially pass-through inner duct having at least one main diameter said central pin having a first end connected to said baseplate and a second free end opposite said first end;
said duct having a helical cavity made in an inner wall of the duct delimiting said duct, the inner wall being open towards an inside of the duct and extending along an entire length of said duct, in such a way as to place in fluid communication the second surface of the baseplate with the second end of said central pin; wherein said inner duct has a secondary diameter smaller than said main diameter and said helical cavity has a diameter greater than the main diameter of the inner duct.

2. The device according to claim 1, wherein said helical cavity has a pitch and profile designed to be coupled with a pitch and profile of a fixing screw that can be screwed into said duct.

3. The device according to claim 1, wherein said baseplate has, at the pin, a central hole passing through and communicating with the duct inside the pin; said first end of said pin being connected to said baseplate and opening externally into said second concave surface through said central hole, while said second end of said central pin is open externally.

4. The device according to claim 1, wherein said inner duct has at least one threaded portion having a pitch and height different from those of the helical cavity, so that it can be coupled with a second threaded screw which may be fitted through said first end of said central pin, adapted to fix a glenosphere.

5. The device according to claim 1, wherein said inner duct has an end portion, placed at the second end of said pin, having a diameter smaller than the main diameter of the duct.

6. The device according to claim 1, wherein a change in diameter of said duct takes place in an intermediate zone between the second surface of the baseplate and the second end of the central pin; said duct having the main greater diameter extending in a portion between the baseplate and said intermediate zone and the secondary smaller diameter extending in the portion between said intermediate zone and the second end of the central pin.

7. The device according to claim 1, wherein said pin has a cylindrical shape and has a corrugated outer surface comprising a plurality of circumferential discharges.

8. The device according to claim 1, wherein said baseplate has at least two peripheral through holes that place said first at least partially convex surface in communication with said second concave surface 22.

9. The device according to claim 1, wherein said first surface has, at least partially, raised wedge-shaped portions.

10. A bone fixing screw insertable in the device according to claim 1 for anchoring a shoulder prosthesis in a reverse configuration, comprising: a cylindrical body, a tip placed at one end of the cylindrical body, a head placed at a second end of the cylindrical body, opposite to the first end, and a thread extending helically along an entire axial extension of said cylindrical body, wherein said head has an outer diameter greater than the outer diameter of said cylindrical body and smaller than the outer diameter of the thread.

11. The bone fixing screw according to claim 10, wherein said head has a cylindrical shape, without protruding portions; said head connecting to said cylindrical body by a tapered portion.

12. The fixing screw according to claim 10, wherein said tip has at least one notch having a cutting edge.

13. An anchoring system for anchoring a shoulder prosthesis in a reverse configuration comprising an anchoring device and a fixing screw, wherein:
the anchoring device comprises:
a baseplate delimited at a bottom of the baseplate by a first at least partially convex surface, adapted to interface with a glenoid, at a top of the baseplate by a second concave surface, opposite the first surface, and peripherally by a third truncated-cone or cylindrical surface,
a central pin, protruding from said first surface, having an axially pass-through inner duct having at least one main diameter, said central pin having a first end connected to said baseplate and a second free end opposite said first end;
said duct having a helical cavity made in an inner wall of the duct delimiting said duct, the inner wall being open towards an inside of the duct and extending along an entire length of said duct, in such a way as to place in fluid communication the second surface of the baseplate with the second end of said central pin; wherein said inner duct has a secondary diameter smaller than said main diameter and said helical cavity has a diameter greater than the main diameter of the inner duct; and
the fixing screw comprises:
a cylindrical body, a tip placed at one end of the cylindrical body, a head placed at a second end of the cylindrical body, opposite to the first end, and a thread extending helically along an entire axial extension of said cylindrical body, wherein said head has an outer diameter greater than the outer diameter of said cylindrical body and smaller than the outer diameter of the thread.

14. The anchoring system according to claim 13, wherein said helical cavity has a pitch, profile and depth designed to be coupled with a pitch, profile and height of the thread of the fixing screw which can be screwed into said duct.

15. The anchoring system according to claim 13, wherein the head of the fixing screw has an outer diameter equal to the secondary diameter of the duct inside the anchoring device.

16. The anchoring system according to claim 13, wherein the outer diameter of the thread of the fixing screw is equal to the diameter of the helical cavity of the anchoring device.
